(19) United States
(12) Patent Application Publication
(10) Pub. No.: US 2002/0044991 A1
(43) Pub. Date: Apr. 18, 2002

(54) Solid preparations having a multicore structure

(76) Inventors: Helmut Auweter, Limburgerhof (DE); Nina Musaeus Jensen, Hellerup (DK); Erik Luddecke, Mutterstadt (DE); Frank Runge, Friedelsheim (DE)

Correspondence Address: Herbert B. Keil, KEIL & WEINKAUF, 1101 Connecticut Ave., N.W., Washington, DC, 20036, US

(21) Appl. No.: 09/929,075

(22) Filed: Aug. 15, 2001

(30) Foreign Application Priority Data
Aug. 30, 2000 (DE) 10042833.9

(51) Int. Cl. A61K 47/00

(52) U.S. Cl. 426/72; 424/439; 426/576

(57) ABSTRACT

The invention relates to solid preparations of at least two active compounds suitable for the food sector and animal feed sector or for pharmaceutical and cosmetic applications having a multicore structure in which at least two cores of a multicore structure have a different chemical composition, a process for their production and the use of these solid preparations to produce food supplements, and as additive to foods, animal feeds, pharmaceutical and cosmetic preparations.

The invention relates to solid preparations of at least two active compounds suitable for the food sector and animal feed sector or for pharmaceutical and cosmetic applications having a multicore structure, in particular carotenoid-containing dry powders, a process for their production and the use of these solid preparations for producing food supplements and as additive to foods, animal feeds, pharmaceutical and cosmetic preparations.

The use of solid preparations, for example mixtures of fat-soluble vitamins and/or carotenoids, whose composition is matched to physiological requirements and in which the individual components are in part present in extreme excess or deficiency, imposes high requirements on formulation. For the user, it is particularly important in this case that, in addition to the desired stability, homogeneous equal distribution of the active compounds is assured in all particles.

A number of methods are disclosed in the patent literature for formulating carotenoids.

Thus, EP-A-0 065 193 and EP-A-0 937 412 describe processes for converting carotenoids into finely divided pulverulent forms.

EP-A-0498 824 discloses a process for grinding carotenoids in a protective-colloid-containing aqueous medium and subsequent conversion of this dispersion into a dry powder.

EP-A-0 410 236 relates to a process for producing colloidal carotenoid preparations by contacting a suspension of a carotenoid in a high-boiling oil with superheated steam, emulsifying this mixture in an aqueous protective colloid solution and subsequent drying.

WO 98/26008 describes a process for producing stable aqueous dispersions and dry powders of xanthophylls.

WO 99/48487 describes preparations of carotenoid mixtures in which the carotenoids originate from natural sources. Owing to the high phospholipid content in these preparations, together with a high viscosity of the oily dispersion, the service properties of this formulation are not always satisfactory.

The abovementioned preparations, when carotenoid mixtures are used, not infrequently encounter problems with stability and bioavailability. In addition, in the case of mixtures having extremely different contents of the individual carotenoids, formation of aggregates among the carotenoids can lead to unwanted inhomogeneous distributions of the active compounds in these preparations. Furthermore, mixtures of dry powders of individual carotenoids also frequently display separation during transport or storage.

It is an object of the present invention, therefore, to propose solid active component preparations or uses in the food sector and animal nutrition section and for pharmaceutical and cosmetic applications, in which, in addition to the desired stability, homogeneous equal distribution of active compounds is ensured in all particles.

We have found that this object is achieved according to the invention by solid preparations of at least two active compounds suitable for the food sector and animal feed sector or for pharmaceutical and cosmetic applications in the form of a multicore structure in which at least two cores of a multicore structure have a different chemical composition.

For the purposes of the invention, the multicore structure is a particle species (secondary particle) having a mean particle size of from 5 to 3000 μm, preferably from 10 to 2500 μm, particularly preferably from 50 to 2000 μm, very particularly preferably from 100 to 1000 μm, in which a further particle species (primary particle), called cores, is embedded in a matrix, the cores having a mean particle size, preferably, of from 0.01 to 1.0 μm, particularly preferably from 0.03 to 0.5 μm, very particularly preferably from 0.05 to 0.2 μm.

Examples of such multicore structures are found, inter alia, in U.S. Pat. No. 5,780,056 and in the diagrams described there and in D. Horn and E. Lüddecke: “Preparation and characterization of nano-sized carotenoid hydrosols” in Fine Particle Science and Technology, 761-775 [E. Pelizzetti (Ed.), Kluwer Academic Publishers, Netherlands, 1996] and H. Auweter et al., Angew. Chem. Int. Ed. 38 (1999) 5, 2188-91.

A feature of the previously known multicore structures is that their primary particles (see above) are identical in composition, that is to say in the case of a mixture, for example of carotenoids and/or vitamins, each core is identical with respect to type and amount of the carotenoid/vitamin individual components present therein.

A feature of the inventive solid preparations is now that they firstly prevent or decrease unwanted interactions between the active compounds within the multicore structure by encapsulation of the individual active compounds, and secondly they permit more flexible organization of the production of user-friendly formulations of active-compound-containing mixtures.

For the purposes of the present invention, active compounds suitable for the food sector and animal nutrition sector or for pharmaceutical and cosmetic applications are the following compounds:

Fat-soluble vitamins, for example the K vitamins, vitamin A and derivatives such as vitamin A acetate, vitamin A propionate or vitamin A palmitate, vitamin D₂ and vitamin D₃ and vitamin E and derivatives. Vitamin E in this context is natural or synthetic α-, β-, γ- or δ-tocopherol, preferably natural or synthetic α-tocopherol, or else is tocotrienol. Vitamin E derivatives are, for example, tocopheryl C₁-C₂₀-acyl esters such as tocopheryl acetate or tocopheryl palmitate.

Water-soluble vitamins, in particular ascorbic acid and its salts such as sodium ascorbate, and vitamin C derivatives such as sodium, calcium or magnesium ascorbyl 2-monophosphate or calcium ascorbyl 2-polyphosphate, calcium pantothenate, panthenol, vitamin B₁ (thiamine), as hydrochloride, nitrate or pyrophosphate, vitamin B₂ (riboflavin) and its phosphates, vitamin B₆ and salts, vitamin B₁₂, biotin, folic acid and folic acid derivatives such as tetrahydrofolic acid, 5-methyltetrahydrofolic acid, 5-formyltetrahydrofolic acid, nicotinic acid and nicotinamide.

Compounds having vitamin character or coenzyme character, for example choline chloride, carnitine, γ-butyrobetaine, lipoic acid, kreatine, ubiquinones, S-methylmethionine, S-adenosylmethionine.

Polyunsaturated fatty acids, for example linleoic acid, linolenic acid, arachidonic acid, eicosapentaenoic acid, docosahexaenoic acid.

Food pigments such as curcumin, carmine or chlorophyll.

carotenoids, not only carotenes but also xanthophylls, for example β-carotene, lycopene, lutein, astaxanthin, zeaxanthin, capsanthin, capsorubin, cryptoxanthin, citranaxanthin, canthaxanthin, bixin, β-apo-4-carotenal, β-apo-8-carotenal and β-apo-8-carotenic esters.

Preferred embodiments of the inventive solid preparations are carotenoid-containing dry powders in the form of the abovementioned multicore structure which comprise at least two of the abovementioned carotenoids, selected from the group consisting of carotenes and xanthophylls.

Particular preference is given to those dry powders in which at least two cores (primary particles) comprise one carotenoid or more than one different carotenoids. In particular in the preparations at least two cores comprise only one representative of the carotenoid class of substances.

The carotenoids present in the cores can be of either natural or synthetic origin. They generally have a purity of at least 80%, preferably greater than 90%, particularly preferably greater than 95%, very particularly preferably greater than 98%, determined by quantitative HPLC analysis.

In the case of carotenoids from natural sources, for example lutein or lycopene, it is possible that these comprise up to 20% of other carotenoids as “impurities”.

Preferred carotenoids which may be mentioned are carotenes such as β-carotene and lycopene or xanthophylls such as astaxanthin, lutein, zeaxanthin and canthaxanthin.

Very particular preference is given to dry powders comprising a mixture of β-carotene, lycopene and lutein.

A dry powder of this type comprises a multicore structure of secondary particles in which at least three primary particles have a different carotenoid composition, in each case one particle species comprising only β-carotene, the second lycopene and the third only lutein.

The content of β-carotene, lycopene and lutein in the inventive dry powders is generally from 0.1 to 50% by weight, preferably from 1 to 35% by weight, particularly preferably from 5 to 25% by weight, very particularly preferably from 8 to 20% by weight, based on the total amount of the formulation.

In the case of the abovementioned ternary combination, the quantitative ratio of the carotenoids present in the dry powder is 1 part of β-carotene, from 0.02 to 20 parts of lycopene and from 0.02 to 20 parts of lutein, preferably 1 part of β-carotene, from 0.1 to 5 parts of lycopene and from 0.1 to 5 parts of lutein, particularly preferably 1 part of β-carotene, from 0.2 to 2 parts of lycopene and from 0.1 to 2 parts of lutein, very particularly preferably 1 part of β-carotene, from 0.3 to 1.2 parts of lycopene and from 0.1 to 0.8 parts of lutein.

In the carotenoid formulations, in particular the abovementioned ternary combination, in addition, the phosphorus content in the formulations is less than 2.0% by weight, advantageously less than 1.0% by weight, preferably less than 0.5% by weight, particularly preferably less than 0.1% by weight, very particularly preferably less than 0.02% by weight, based on the total amount of the mixture of β-carotene, lycopene and lutein.

The low phosphorus content is at the same time associated with a small amount of phospholipids, which improves the service properties of the dry powders, for example the flowability in oily dispersions particularly at low temperatures.

The carotenoid formulations can comprise, in their secondary particles, in addition to the above-described carotenoid-containing cores, other primary particles whose active compounds do not originate from the carotenoid class of substances. These are preferably vitamin-containing primary particles.

In the inventive preparations, in addition, the primary particles have a core/shell structure in which the active-compound-containing core is surrounded by a protective colloid.

Suitable protective colloids are either electrically charged polymers (polyelectrolytes) or neutral polymers. Typical examples are, inter alia, gelatin, such as beef gelatin, pig gelatin or fish gelatin, starch, dextrin, plant proteins, such as soy proteins, which may be hydrolyzed, pectin, guar gum, xanthan, gum arabic, casein, caseinate or mixtures thereof. However, use may also be made of polyvinyl alcohol, polyvinylpyrrolidone, methyl cellulose, carboxymethyl cellulose, hydroxypropyl cellulose, flake shellac and alginates. For more details see R. A. Morton, Fat Soluble Vitamins, Intern. Encyclopedia of Food and Nutrition, Vol. 9, Pergamon Press 1970, pp. 128-131.

Preferred protective colloids are compounds selected from the group consisting of gelatin, such as beef gelatin, pig gelatin and fish gelatin, plant proteins, pectin, casein, caseinate, gum arabic and shellac. Protective colloids which are particularly preferably used are aqueous solutions of gelatin, pectin, casein, caseinate, gum arabic and/or fish gelatin.

To increase the mechanical stability of the dry powder, it is expedient to add to the colloid a plasticizer, such as sugars or sugar alcohols, for example sucrose, glucose, lactose, invert sugar, sorbitol, mannitol or glycerol, or else polymers such as polyvinyl alcohol or polyvinylpyrrolidone. Plasticizers preferably used are sucrose, sorbitol and lactose.

The ratio of protective colloid and plasticizer to active compound is generally chosen so that a solid preparation is obtained which comprises from 0.1 to 50% by weight of at least two active compounds, from 10 to 50% by weight, preferably from 15 to 35% by weight, of a protective colloid and from 20 to 70% by weight, preferably from 30 to 60% by weight, of a plasticizer, all percentages being based on the dry matter of the formulation and the total of the percentages of the individual components being 100%.

To increased the stability of the active compounds to oxidative degradation, it can be advantageous to add from 0 to 10% by weight, preferably from 0.5 to 7.5% by weight, based on the dry matter of the formulation, of one or more stabilizers, such as α-tocopherol, tert-butylated hydroxytoluene, tert-butylated hydroxyanisole, ascorbic acid or ethoxyquins.

In addition, emulsifiers can be used, for example ascorbyl palmitate, polyglycerol fatty acid esters, sorbitol fatty acid esters, propylene glycol fatty acid esters or lecithin at a concentration of from 0 to 200% by weight, preferably from 5 to 150% by weight, particularly preferably from 10 to 80% by weight, based on the active compounds used.

In some circumstances it can also be advantageous to use in addition a physiologically permissible oil, for example sesame seed oil, corn oil, cotton seed oil, soybean oil or peanut oil, and esters of medium-chain plant fatty acids at a concentration of from 0 to 500% by weight, preferably from 10 to 300% by weight, particularly preferably from 20 to 100% by weight, based on the active compounds.

The matrix present in the multicore structure is generally formed from a physiologically acceptable polymeric material. Preferably it is composed of at least one of the abovementioned protective colloids, possibly in combination with the above-described formulation aids, such as plasticizers, antioxidants and/or emulsifiers. The matrix can also comprise at least one water-soluble vitamin.

The invention also relates to a process for producing the above-described solid preparations by drying an aqueous suspension comprising at least two active compounds which are suitable for the food sector and animal feed sector or for pharmaceutical and cosmetic applications in the form of nanoparticulate particles, which comprises at least two of the nanoparticulate particles having a different chemical composition. Active compounds here are the compounds already mentioned at the outset.

In a preferred embodiment of the process, the active compounds are at least two carotenoids, in which case, particularly preferably, at least two of the nanoparticulate particles comprise one or more different carotenoids.

In a very particularly preferred process variant, at least two of the nanoparticulate particles comprise only one representative from the carotenoid class of substances.

For reasons of stability it is advantageous in this case if the active compounds are present in the form of protective-colloid-stabilized nanoparticulate particles which have a mean particle size of, preferably, from 0.01 to 1.0 μm, particularly preferably from 0.03 to 0.5 μm, very particularly preferably from 0.05 to 0.2 μm.

The active compounds, in particular the carotenoids, used to produce the inventive preparations can be used in the form of very finely ground crystals, or preferably in the form of preprepared dry powders. These dry powders each comprise nanoparticulate particles of the individual carotenoids and may be produced by grinding or micronizing individual active compounds. Examples of these may be found, inter alia, in EP-A-0 065 193, EP-A-0 937 412 and in WO 91/06292.

By redispersing the starting formulations in aqueous solutions and converting the dispersion again into a dry powder by processes known per se, for example spray-drying or spray-cooling, with or without addition of dusting powders to avoid agglomeration, the novel inventive preparations having the multicore structures described at the outset may be obtained.

Details on spray-drying or spray-cooling may be found, inter alia, in WO 91/06292.

The inventive carotenoid formulations are suitable, inter alia, as additive for coloring food preparations, in particular drink preparations, as agent for producing pharmaceutical and cosmetic preparations and for producing food supplement preparations in the human and animal sectors.

Thus, drinks may be colored, for example, by using the inventive water-dispersible dry powders in which are present mixtures of β-carotene, lycopene and lutein at the concentrations already mentioned above.

It is also possible to use dry powders which comprise the inventive carotenoid combinations to enrich milk products such as yogurt, flavored milk drinks or ice cream, or milk pudding powders, baking mixes and confectionery products, for example fruit gums.

The invention also relates to food supplements, animal feeds, foods and pharmaceutical and cosmetic preparations comprising the above-described preparations, in particular carotenoid formulations of mixtures of β-carotene, lycopene and lutein.

Food supplement preparations and pharmaceutical preparations which comprise the inventive dry powders are, inter alia, tablets, sugar-coated tablets and hard and soft gelatin capsules. Preferred food supplement preparations are tablets into which the dry powders are coincorporated, and soft gelatin capsules in which the carotenoid-containing multicore structures are present as oily suspension in the capsules. The carotenoid content in these capsules is from 0.5 to 20 mg of β-carotene, from 0.5 to 20 mg of lycopene and 0.5 to 20 mg of lutein, preferably from 1 to 15 mg of β-carotene, from 1 to 15 mg of lycopene and from 1 to 10 mg of lutein, particularly preferably from 2 to 10 mg of β-carotene, from 2 to 10 mg of lycopene and from 1 to 5 mg of lutein.

In the examples below, production of the inventive dry powders will be described in more detail.

### EXAMPLE 1

500 g of β-carotene-containing dry powder having a β-carotene content of 20% by weight, 500 g of lycopene-containing dry powder having a lycopene content of 10% by weight and 200 g of lutein-containing dry powder having a lutein-content of 10% by weight (all dry powders produced according to EP-B-0 065 193) were redispersed in 1800 ml of water at 65° C. with stirring. After the powder matrix was completely dissolved, the viscosity of the dispersion was set to a value of approximately 180 cP (measured at 65° C.) by adding water. The dispersion was then converted into a powder by spray-cooling and subsequent drying. The following carotenoid content was determined in the dry powder by HPLC:

| | | |
|---|---|---|
| β-Carotene: | 5.3% | by weight |
| Lycopene: | 3.0% | by weight |
| Lutein: | 1.1% | by weight |
| Total carotenoid content: | 9.4% | by weight |

### EXAMPLE 2

In a similar manner to Example 1, 575 g of β-carotene-containing dry powder having a β-carotene content of 20% by weight, 500 g of lycopene-containing dry powder having a lycopene content of 10% by weight and 200 g of lutein-containing dry powder having a lutein content of 10% by weight were redispersed in 1900 ml of water and then dried. A dry powder of the following carotenoid composition was obtained:

| | | |
|---|---|---|
| β-Carotene: | 5.7 | %by weight |
| Lycopene: | 2.9% | by weight |
| Lutein: | 1.1% | by weight |
| Total carotenoid content: | 9.7% | by weight |

### EXAMPLE 3

In a similar manner to Example 1, 700 g of β-carotene-containing dry powder having a β-carotene content of 20% by weight and 600 g of lutein-containing dry powder having a lutein content of 10% by weight were redispersed in 1800 ml of water and then dried. A dry powder of the following carotenoid composition was obtained:

| | | |
|---|---|---|
| β-Carotene: | 7.1% | by weight |
| Lutein: | 3.5% | by weight |
| Total carotenoid content: | 10.6% | by weight |

We claim:

1. A solid preparation of at least two active compounds suitable for the food sector and animal feed sector or for pharmaceutical and cosmetic applications in the form of a multicore structure in which at least two cores of a multicore structure have a different chemical composition.

2. A solid preparation as claimed in claim 1, wherein the multicore structure is a particle species having a mean particle size of from 5 to 3000 μm in which the cores are embedded in a matrix.

3. A solid preparation as claimed in one of claims 1 or 2 in which the cores have a mean particle size of from 0.01 to 1.0 μm.

4. A solid preparation as claimed in one of claims 1 to 3, which is a carotenoid-containing dry powder of at least two carotenoids.

5. A carotenoid-containing dry powder as claimed in claim 4, wherein at least two cores comprise one or more different carotenoids.

6. A carotenoid-containing dry powder as claimed in one of claims 4 or 5, wherein at least two cores comprise only one representative of the carotenoid class of substances.

7. A carotenoid-containing dry powder as claimed in one of claims 4 to 6, wherein the carotenoids are a selection from the group of the carotenes and xanthophylls.

8. A carotenoid-containing dry powder as claimed in one of claims 4 to 7, comprising β-carotene, lycopene and lutein.

9. A carotenoid-containing dry powder as claimed in claim 8, comprising 1 part by weight of β-carotene, from 0.02 to 20 parts by weight of lycopene and from 0.02 to 20 parts by weight of lutein.

10. A carotenoid-containing dry powder as claimed in one of claims 4 to 9, having a carotenoid content of from 0.1 to 50% by weight, based on the total amount of the dry powder.

11. A process for producing solid preparations defined according to claim 1 by drying an aqueous suspension comprising at least two active compounds which are suitable for the food sector and animal feed sector or for pharmaceutical and cosmetic applications in the form of nanoparticulate particles, which comprises at least two of the nanoparticulate particles having a different chemical composition.

12. A process as claimed in claim 11, wherein the active compounds are at least two carotenoids.

13. A process as claimed in one of claims 11 or 12, wherein at least two of the nanoparticulate particles comprise one or more different carotenoids.

14. A process as claimed in one of claims 11 to 13, wherein at least two of the nanoparticulate particles comprise only one representative of the carotenoid class of substances.

15. A process as claimed in one of claims 11 to 14, wherein the active compounds are present in the form of protective-colloid-stabilized nanoparticulate particles.

16. A process as claimed in one of claims 11 to 15, wherein the nanoparticulate particles have a size of from 0.01 to 1.0 μm.

17. The use of the solid preparation defined according to one of claims 1 to 10 for producing food supplements and as additive to foods, animal feeds, pharmaceutical and cosmetic preparations.

18. The use as claimed in claim 17 for producing soft gelatin capsules.

19. A food supplement, food, animal feed and pharmaceutical and cosmetic preparation comprising carotenoid-containing preparations defined according to one of claims 1 to 10.
